(19) United States
(12) Patent Application Publication
(10) Pub. No.: US 2023/0134313 A1
(43) Pub. Date: May 4, 2023

(54) SYSTEMS AND METHODS FOR DETECTION OF LOW-ABUNDANCE MOLECULAR BARCODES FROM A SEQUENCING LIBRARY

(71) Applicant: 10x Genomics, Inc., Pleasanton, CA (US)

(72) Inventors: Daniel P. Riordan, Hillsborough, NC (US); Maria Alexis, Oakland, CA (US); Joseph Glenn Arthur, Oakland, CA (US); Andrew John Hill, Oakland, CA (US)

(21) Appl. No.: 18/090,903

(22) Filed: Dec. 29, 2022

Related U.S. Application Data
(63) Continuation of application No. PCT/US2021/040181, filed on Jul. 1, 2021.
(60) Provisional application No. 63/047,891, filed on Jul. 2, 2020.

(51) Int. Cl. C12Q 1/6869; G16B 30/00

(52) U.S. Cl. CPC C12Q 1/6869; G16B 30/00

(57) ABSTRACT

In one aspect, a method for filtering out erroneous sequence reads from a genomic sequence dataset, is disclosed. The genomic sequence dataset is received by one or more processors. The dataset is comprised of a plurality of fragment sequence reads, each with an associated barcode sequence and a unique identifier sequence. A threshold value for filtering out select fragment sequence reads from the genomic sequence dataset is determined by the one or more processors. The threshold value is a number of fragment sequence reads in the genomic sequence dataset with the same unique identifier sequence. Fragment sequence reads with the same unique identifier sequence occurring at less than the threshold value in the genomic sequence dataset is filtered out by the one or more processors. A filtered genomic sequence dataset is generated by the one or more processors.

### APPLICATIONS FOR CLAIM OF PRIORITY

The present application is a continuation application under 35 U.SC. 111(a) of PCT/US2021/040181, filed Jul. 1, 2021, entitled “SYSTEMS AND METHODS FOR DETECTION OF LOW-ABUNDANCE MOLECULAR BARCODES FROM A SEQUENCING LIBRARY,” which claims priority to U.S. Provisional Patent Application No. 63/047,891, filed on Jul. 2, 2020, entitled “SYSTEMS AND METHODS FOR DETECTION OF LOW-ABUNDANCE MOLECULAR BARCODES FROM A SEQUENCING LIBRARY,” which applications are herein incorporated by reference in their entirety for all purposes.

### FIELD

This description is generally directed towards systems and methods for detecting low-abundance molecular barcodes in a sequencing library using multi-modal droplet-based single cell genomic sequencing technologies. More specifically, systems and methods for eliminating sequencing workflow artifacts with no biological meaning, such as chimeric molecules arising from PCR amplification, prior to downstream sequence data analysis.

### BACKGROUND

Most DNA sequencing libraries undergo PCR amplification before being sequenced. A byproduct of the amplification reaction is that “chimeric” and other artifact molecules can be created, in which parts of two distinct molecules from the original library are present in a single amplified molecule. Following sequencing, it is desirable to remove these chimeric sequences since they are irrelevant to the original library and can confound downstream analysis and lower the specificity with which molecules of interest are detected.

Conventionally, chimeric molecules have been detected at the sequence level, i.e., by determining that portions of the sequence arise from two or more separate reference sequences, e.g., different gene transcripts or chromosomes. However, this may require specialized alignment methods and is otherwise not ideal for certain targeted sequencing libraries (i.e., libraries where less than about 100 base pairs of the insert are sequenced).

As such, there is a need for systems and methods that can eliminate sequencing workflow artifacts, such as chimeric molecules, prior to downstream sequence data analysis to allow for the detection of low-abundance molecular barcodes in a targeted sequencing library. Such systems and methods can provide filtered sequencing data sets that affords greater confidence that molecules detected by few reads in the data set are in fact real and are not the result of artifacts created during PCR amplification.

### SUMMARY

In one aspect, a method for filtering out erroneous sequence reads from a genomic sequence dataset, is disclosed. The genomic sequence dataset is received by one or more processors. The dataset is comprised of a plurality of fragment sequence reads, each with an associated barcode sequence and a unique identifier sequence.

A threshold value for filtering out select fragment sequence reads from the genomic sequence dataset is determined by the one or more processors. The threshold value is a number of fragment sequence reads in the genomic sequence dataset with the same unique identifier sequence.

Fragment sequence reads with the same unique identifier sequence occurring at less than the threshold value in the genomic sequence dataset is filtered out by the one or more processors. A filtered genomic sequence dataset is generated by the one or more processors.

In another aspect, a system for filtering out erroneous sequence reads from a genomic sequence dataset, is disclosed. The system includes a data store and a computing device. The data store is configured to store the genomic sequence dataset comprising a plurality of fragment sequence reads, each with an associated barcode sequence and a unique identifier sequence.

The computing device is communicatively connected to the data store and is comprised of a unique molecule filtering engine. The unique identifier engine is configured to receive the genomic sequence dataset, determine a threshold value for filtering out select fragment sequence reads from the genomic sequence dataset, filter fragment sequence reads with the same unique identifier sequence occurring at less than the threshold value in the genomic sequence dataset, and generate a filtered genomic sequence dataset. The threshold value is a number of fragment sequence reads in the genomic sequence dataset with the same unique identifier sequence.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIGS. 1A and 1B are schematic illustrations of non-limiting examples of the sequencing workflow for using single cell targeted gene expression sequencing analysis to generate sequencing data for analyzing the expression profile of targeted genes of interest, in accordance with various embodiments.

FIG. 2 is an illustration of a chimeric molecule (or chimera), in accordance with various embodiments.

FIG. 3 is an illustration of PCR amplification effects on pooled libraries with chimeric molecules, in accordance with various embodiments.

FIG. 4 is a frequency distribution plot of fragment sequence reads associated with each unique molecular identifier (UMI) generated from a sequencing dataset, in accordance with various embodiments.

FIG. 5 is a schematic illustration of a non-limiting example of the sequencing data analysis workflow for analyzing single cell targeted gene expression sequencing data, in accordance with various embodiments

FIG. 6 is a flowchart illustrating a non-limiting example method for filtering out erroneous sequence reads from a genomic sequence dataset, in accordance with various embodiments, in accordance with various embodiments.

FIG. 7 is a diagram illustrating a non-limiting example system for filtering out erroneous sequence reads from a genomic sequence dataset, in accordance with various embodiments.

FIG. 8 is a block diagram that illustrates a computer system, upon which embodiments, or portions of the embodiments, may be implemented, in accordance with various embodiments.

FIG. 9 shows the performance of two molecule filtering strategies, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

This specification describes various exemplary embodiments of methods for eliminating sequencing workflow artifacts, such as chimeric molecules, prior to downstream sequence data analysis to allow for the detection of low-abundance molecular barcodes in a targeted sequencing library. Such systems and methods can provide filtered sequencing data sets that affords greater confidence that molecules detected by few reads in the data set are in fact real and are not the result of artifacts created during PCR amplification.

It should be appreciated, however, that although the systems and methods disclosed herein refer to their application in targeted gene expression analysis workflows, they are equally applicable to other analogous fields.

In various embodiments, a computer program product can include instructions to receive an output file for a genomic sequence dataset, the output file comprised of a plurality of fragment sequence reads, each with an associated barcode sequence and a unique identifier sequence (i.e., unique molecular identifier or UMI); instructions to determine a threshold value for filtering out select fragment sequence reads from the genomic sequence dataset; instructions for filtering out fragment sequence reads with the same unique identifier sequence occurring less than the threshold value in the dataset; and instructions to generate an updated output file including a filtered genomic sequence dataset.

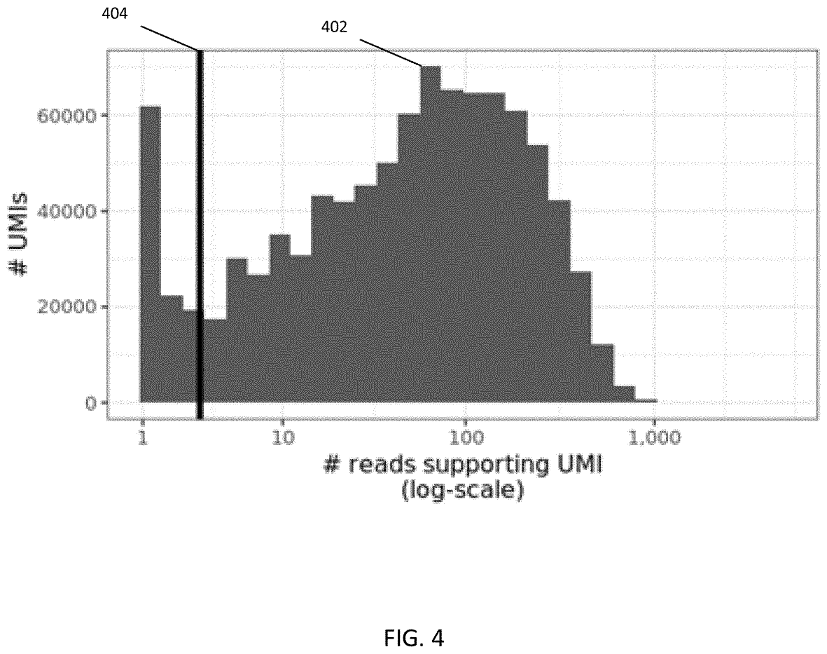

FIG. 4 is a frequency distribution plot of fragment sequence reads associated with each unique molecular identifier (UMI) generated from a genomic sequencing dataset, in accordance with various embodiments. As shown in FIG. 4, a threshold 404 value of 3 reads can be established on the frequency distribution plot 402 and unique UMIs with (strictly) less than 3 reads are discarded to remove sequencing workflow artifacts, such as chimeric molecules from the genomic sequencing dataset.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

All publications mentioned herein are incorporated herein by reference for the purpose of describing and disclosing devices, compositions, formulations and methodologies which are described in the publication and which might be used in connection with the present disclosure.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, un-recited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, pharmacology and toxicology, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina, the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition to, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject.

In some examples, such systems provide “sequencing reads” (also referred to as “fragment sequence reads” or “reads” herein). A read may include a string of nucleic acid bases corresponding to a sequence of a nucleic acid molecule that has been sequenced. In some situations, systems and methods provided herein may be used with proteomic information.

Methods of processing and sequencing nucleic acids in accordance with the methods and systems described in the present application are also described in further detail in Published US Application Nos. US20140227684A1, US20140228255A1, US20140378345A1, US20150376609A1, and US20190136316A1, which are herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing reads.

As used herein, the term “cell barcode” refers to any barcodes that have been determined to be associated with a cell, as determined by a “cell calling” step within various embodiments of the disclosure.

As used herein, the term “Gel bead-in-EMulsion” or “GEM” refers to a droplet containing some sample volume and a barcoded gel bead, forming an isolated reaction volume. When referring to the subset of the sample contained in the droplet, the term “partition” may also be used. In various embodiments within the disclosure, the term barcode can refer to a GEM containing a gel bead that carries many DNA oligonucleotides with the same barcode, whereas different GEMs have different barcodes.

As used herein, the term “GEM well” or “GEM group” refers to a set of partitioned cells (i.e., Gel beads-in-Emulsion or GEMs) from a single 10× Chromium™ Chip channel. One or more sequencing libraries can be derived from a GEM well.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches. In various embodiments within the disclosure, the term adapter can refer to customized strands of nucleic acid base pairs created to bind with specific nucleic acid sequences, e.g., sequences of DNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “macromolecule” or “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “sample,” as used herein, generally refers to a “biological sample” of a subject. The sample may be obtained from a tissue of a subject. The sample may be a cell sample. A cell may be a live cell. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. In some embodiments, the term “sample” can refer to a cell or nuclei suspension extracted from a single biological source (blood, tissue, etc.).

The sample may comprise any number of macromolecules, for example, cellular macromolecules. The sample may be or may include one or more constituents of a cell, but may not include other constituents of the cell. An example of such cellular constituents is a nucleus or an organelle. The sample may be or may include DNA, RNA, organelles, proteins, or any combination thereof. The sample may be or include a chromosome or other portion of a genome. The sample may be or may include a bead (e.g., a gel bead) comprising a cell or one or more constituents from a cell, such as DNA, RNA, a cell nucleus, organelles, proteins, or any combination thereof, from the cell. The sample may be or may include a matrix (e.g., a gel or polymer matrix) comprising a cell or one or more constituents from a cell, such as DNA, RNA, nucleus, organelles, proteins, or any combination thereof, from the cell.

As used herein, the term “PCR duplicates” refers to duplicates created during PCR amplification. During PCR amplification of the fragments, each unique fragment that is created may result in multiple read-pairs sequenced with near identical barcodes and sequence data. These duplicate reads are identified computationally, and collapsed into a single fragment record for downstream analysis.

As used herein, the term “artifacts” or “PCR artifacts” refers to any unintended genomic sequence segments or molecules that are created during PCR amplification, i.e., sequence segments or molecules not present in the input material.

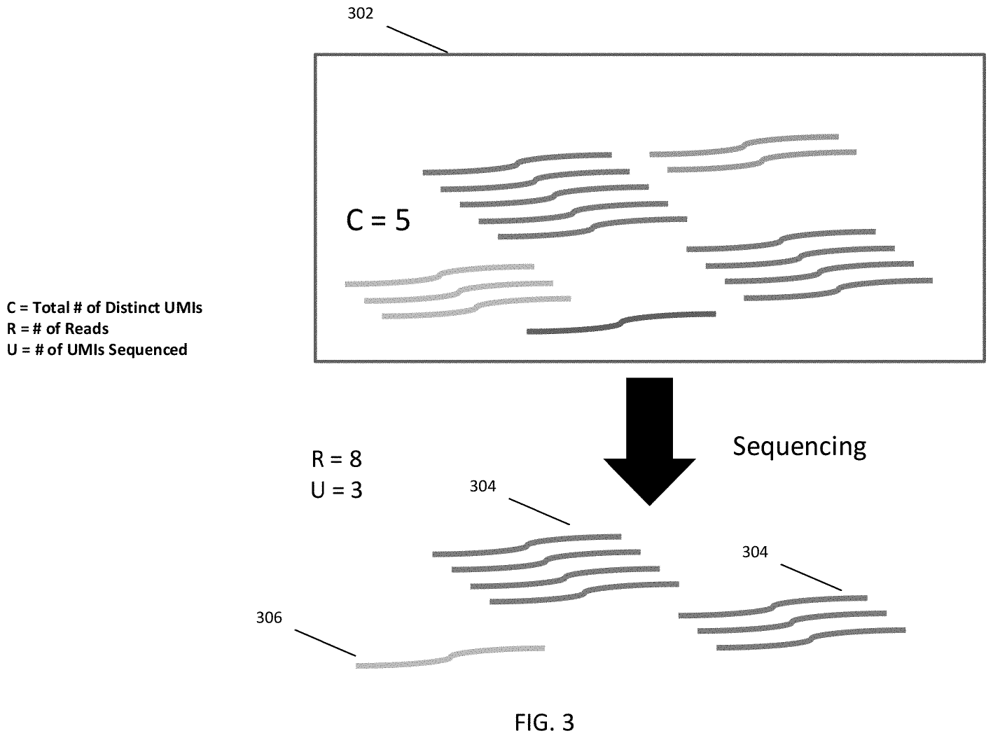

As used herein, the term “chimera molecule” or “chimeric molecule,” as illustrated in FIG. 3, refers to genomic sequences formed from two or more distinctly different genomic sequences, e.g., different gene sequences, originating from separate physical molecules but joined together, typically, during PCR amplification.

As used herein, the term “unique molecular identifier” or “UMI” refers to a genomic sequence segment or molecular tag that is attached to a DNA or RNA fragment prior to PCR amplification. After sequencing, they are used to distinguish sequenced reads from unique DNA or RNA fragments versus PCR duplicates.

As used herein, the term “read data” refers to raw genomic data from sequenced DNA.

As used herein, the term “read-pair” refers to the read data sequenced from one molecule. This can include read1, read2, and the barcode sequence read.

As used herein, the term “sequencing run” refers to a flowcell containing data from one sequencing instrument run. The sequencing data can be further addressed by lane and by one or more sample indices.

### Single Cell Sequencing and Data Analysis Workflows

### Single Cell Targeted Gene Expression Sequencing Workflow

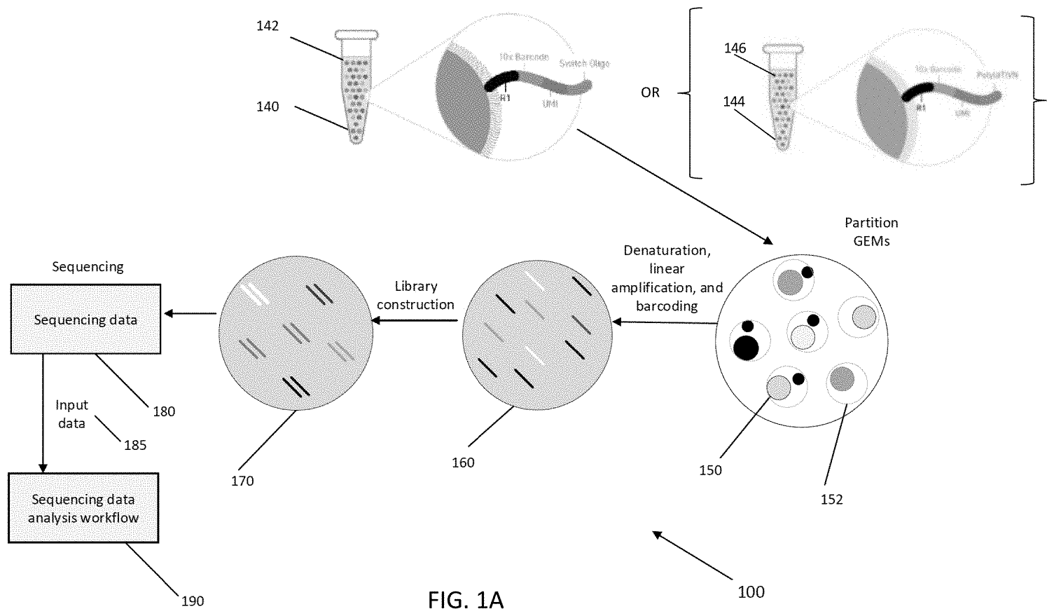

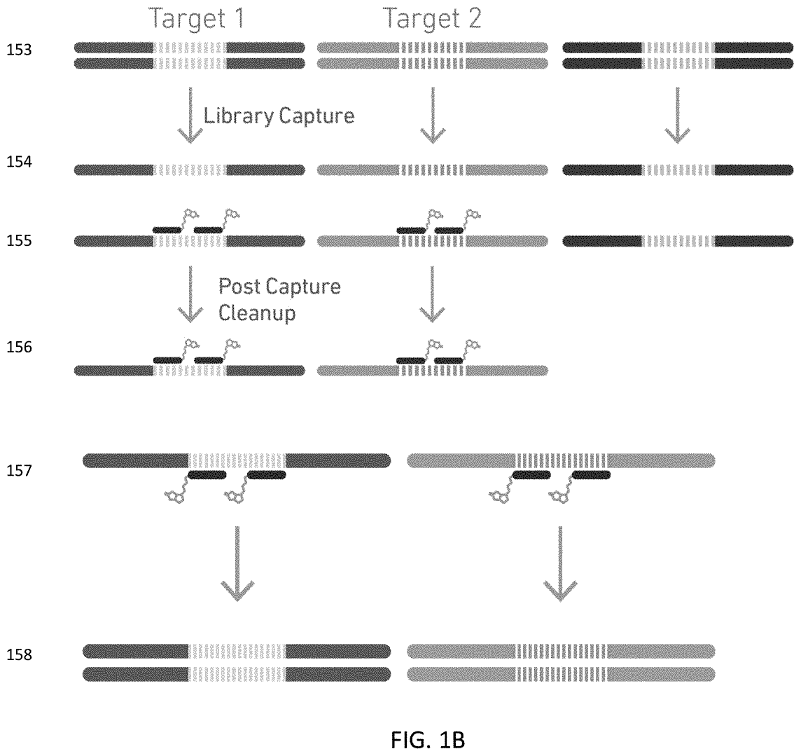

In accordance with various embodiments, a general schematic workflow is provided in FIG. 1 to illustrate a non-limiting example process for using single cell sequencing technology to generate sequencing data. Such sequencing data can be used for targeted gene expression analysis in accordance with various embodiments. The workflow can include various combinations of features, whether it be more or less features than that illustrated in FIG. 1. As such, FIG. 1 simply illustrates one example of a possible workflow.

GEM Generation

The workflow 100 provided in FIG. 1A begins with Gel beads-in-EMulsion (GEMs) generation. The bulk cell suspension containing the cells is mixed with a gel beads solution 140 or 144 containing a plurality of individually barcoded gel beads 142 or 146. In various embodiments, this step results in partitioning the cells into a plurality of individual GEMs 150, each including a single cell, and a barcoded gel bead 142 or 146. This step also results in a plurality of GEMs 152, each containing a barcoded gel bead 142 or 146 but no cell. Detail related to GEM generation, in accordance with various embodiments disclosed herein, is provided below. Further details can be found in U.S. Pat. Nos. 10,343,166 and 10,583,440, US Published Application Nos. US20180179590A1, US20190367969A1, US20200002763A1, and US20200002764A1, and Published International PCT Application No. WO 2019/040637, each of which is incorporated herein by reference in its entirety.

In various embodiments, GEMs can be generated by combining barcoded gel beads, individual cells, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 142 or 146 of the various embodiments herein may include a gel bead attached to oligonucleotides containing (i) an Illumina® P5 sequence (adapter sequence), (ii) a 16 nucleotide (nt) 10× Barcode, and (iii) a Read 1 (Read 1N) sequencing primer sequence. It is understood that other adapter, barcode, and sequencing primer sequences can be contemplated within the various embodiments herein.

In various embodiments, GEMS are generated by partitioning the cells using a microfluidic chip. To achieve single cell resolution per GEM, the cells can be delivered at a limiting dilution, such that the majority (e.g., ˜90-99%) of the generated GEMs do not contain any cells, while the remainder of the generated GEMs largely contain a single cell.

Barcoding RNA Molecules or Fragments

The workflow 100 provided in FIG. 1A further includes lysing the cells and barcoding the RNA molecules or fragments for producing a plurality of uniquely barcoded single-stranded nucleic acid molecules or fragments (such as single-stranded DNA fragments or single-stranded cDNA molecules or fragments). Upon generation of the GEMs 150, the gel beads 142 or 146 can be dissolved releasing the various oligonucleotides of the embodiments described above, which are then mixed with the RNA molecules or fragments resulting in a plurality of uniquely barcoded single-stranded nucleic acid molecules or fragments 160 following a nucleic acid extension reaction, e.g., reverse transcription of mRNA to cDNA, within the GEMs 150. Detail related to generation of the plurality of uniquely barcoded single-stranded nucleic acid molecules or fragments 160, in accordance with various embodiments disclosed herein, is provided below.

In various embodiments, upon generation of the GEMs 150, the gel beads 142 or 146 can be dissolved, and oligonucleotides of the various embodiments disclosed herein, containing a capture sequence, e.g., a poly(dT) sequence or a template switch oligonucleotide (TSO) sequence, a unique molecular identifier (UMI), a unique 10× Barcode, and a Read 1 sequencing primer sequence can be released and mixed with the RNA molecules or fragments and other reagents or a combination of biochemical reagents (e.g., a master mix necessary for the nucleic acid extension process). Denaturation and a nucleic acid extension reaction, e.g., reverse transcription, within the GEMs can then be performed to produce a plurality of uniquely barcoded single-stranded nucleic acid molecules or fragments 160. In various embodiments herein, the plurality of uniquely barcoded single-stranded nucleic acid molecules or fragments 160 can be 10× barcoded single-stranded nucleic acid molecules or fragments. In one non-limiting example of the various embodiments herein, a pool of ˜750,000, 10× barcodes are utilized to uniquely index and barcode nucleic acid molecules derived from the RNA molecules or fragments of each individual cell.

Accordingly, the in-GEM barcoded nucleic acid products of the various embodiments herein can include a plurality of 10× barcoded single-stranded nucleic acid molecules or fragments that can be subsequently removed from the GEM environment and amplified for library construction, including the addition of adaptor sequences for downstream sequencing. In one non-limiting example of the various embodiments herein, each such in-GEM 10× barcoded single-stranded nucleic acid molecule or fragment can include a unique molecular identifier (UMI), a unique 10× barcode, a Read 1 sequencing primer sequence, and a fragment or insert derived from an RNA fragment of the cell, e.g., cDNA from an mRNA via reverse transcription. Additional adaptor sequence may be subsequently added to the in-GEM barcoded nucleic acid molecules after the GEMs are broken.

In various embodiments, after the in-GEM barcoding process, the GEMs 150 are broken and pooled barcoded nucleic acid molecules or fragments are recovered. The 10× barcoded nucleic acid molecules or fragments are released from the droplets, i.e., the GEMs 150, and processed in bulk to complete library preparation for sequencing, as described in detail below. In various embodiments, following the amplification process, leftover biochemical reagents can be removed from the post-GEM reaction mixture. In various embodiments, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

Library Construction

The workflow 100 provided in FIG. 1A further includes a library construction step. In the library construction step of workflow 100, a library 170 containing a plurality of double-stranded DNA molecules or fragments are generated. These double-stranded DNA molecules or fragments can be utilized for completing the subsequent sequencing step. Detail related to the library construction, in accordance with various embodiments disclosed herein, is provided below.

In accordance with various embodiments disclosed herein, an Illumina® P7 sequence and P5 sequence (adapter sequences), a Read 2 (Read 2N) sequencing primer sequence, and a sample index (SI) sequence(s) (e.g., i7 and/or i5) can be added during the library construction step via PCR to generate the library 170, which contains a plurality of double stranded DNA fragments. In accordance with various embodiments herein, the sample index sequences can each comprise of one or more oligonucleotides. In various embodiments, the sample index sequences can each comprise of four to eight or more oligonucleotides. In various embodiments, when analyzing the single cell sequencing data for a given sample, the reads associated with all four of the oligonucleotides in the sample index can be combined for identification of a sample. Accordingly, in one non-limiting example, the final single cell gene expression analysis sequencing libraries contain sequencer compatible double-stranded DNA fragments containing the P5 and P7 sequences used in Illumina® bridge amplification, sample index (SI) sequence(s) (e.g., i7 and/or i5), a unique 10× barcode sequence, and Read 1 and Read 2 sequencing primer sequences.

Various embodiments of single cell sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

Targeted Gene Enrichment by Hybridization Capture

FIG. 1B depicts an example of a workflow for generating a targeted sequencing library using a hybridization capture approach. As shown, step 153 starts with obtaining a library of double stranded barcoded nucleic acid molecules from single cells (e.g., by partitioning single cells into droplets or wells with barcoding reagents including beads having nucleic acid barcode molecules) is denatured to provide single stranded molecules in step 154. To generate a targeted library using the single stranded molecules, a plurality of oligonucleotide probes designed to cover a panel of selected genes is provided. Each gene in the panel is represented by a plurality of labeled (e.g., biotinylated) oligonucleotide probes, which is allowed to hybridize to the single stranded molecules in step 155 to enrich for genes of interest (e.g. Target 1 and Target 2). To allow for capture, step 155 further includes the addition of supports (e.g., beads) that comprise a molecule having affinity for the labels on each labeled oligonucleotide probe. In various embodiments, the oligonucleotide label comprises biotin and the supports comprise streptavidin beads. Following hybridization capture, cleanup steps 156 and 157 are performed (e.g., one or more washing steps to remove unhybridized or off-target library fragments). Captured library fragments are then subjected to nucleic acid extension/amplification to generate a final targeted library for sequencing in step 158. This workflow allows the generation of targeted libraries from gene expression assays. In general, this workflow may be used to enrich any library of fragments having inserts or targets (light gray bar regions) that represent genes, e.g., cDNA transcribed from mRNA of single cells. It should be appreciated, however, that although the description above describes targeted gene enrichment through the use of hybridization capture probes, the methods disclosed herein can also work with other targeted gene enrichment techniques.

Sequencing

The workflow 100 provided in FIG. 1 further includes a sequencing step. In this step, the library 170 can be sequenced to generate a plurality of sequencing data 180. The fully constructed library 170 can be sequenced according to a suitable sequencing technology, such as a next-generation sequencing protocol, to generate the sequencing data 180. In various embodiments, the next-generation sequencing protocol utilizes the Illumina® sequencer for generating the sequencing data. It is understood that other next-generation sequencing protocols, platforms, and sequencers such as, e.g., MiSeq™, NextSeq™ 500/550 (High Output), HiSeq 2500™ (Rapid Run), HiSeq™ 3000/4000, and NovaSeq™, can be also used with various embodiments herein.

Sequencing Data Input and Data Analysis Workflow

The workflow 100 provided in FIG. 1 further includes a sequencing data analysis workflow 190. With the sequencing data 180 in hand, the data can then be output, as desired, and used as an input data 185 for the downstream sequencing data analysis workflow 190 for targeted gene expression analysis, in accordance with various embodiments herein. Sequencing the single cell libraries produces standard output sequences (also referred to as the “sequencing data”, “sequence data”, or the “sequence output data”) that can then be used as the input data 185, in accordance with various embodiments herein. The sequence data contains sequenced fragments (also interchangeably referred to as “fragment sequence reads”, “sequencing reads” or “reads”), which in various embodiments include RNA sequences of the targeted RNA fragments containing the associated 10× barcode sequences, adapter sequences, and primer oligo sequences.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. One example of a software tool that can process and input the sequencing data for downstream data analysis workflow is the cellranger-atac mkfastq tool within the Cell Ranger™ Targeted Gene Expression analysis pipeline. It is understood that, various systems and methods with the embodiments herein are contemplated that can be employed to independently analyze the inputted single cell targeted gene expression analysis sequencing data for studying cellular gene expression, in accordance with various embodiments.

### Single Cell Gene Expression Data Analysis Workflow

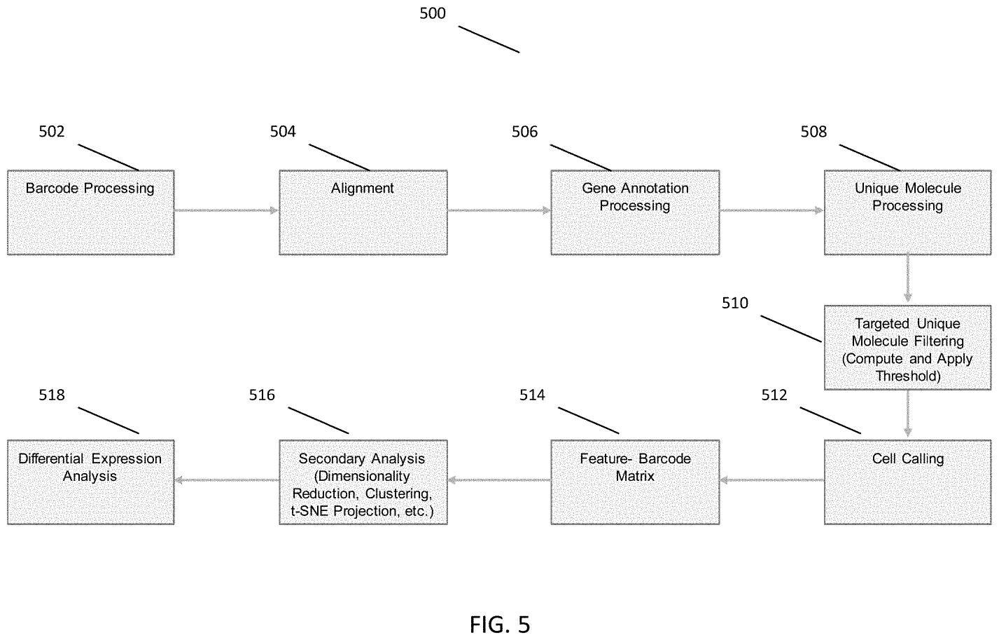

In accordance with various embodiments, a general schematic workflow is provided in FIG. 5 to illustrate a non-limiting example process of a sequencing data analysis workflow for analyzing the single cell targeted gene expression sequencing data. The workflow can include various combinations of features, whether it be more or less features than that illustrated in FIG. 5. As such, FIG. 5 simply illustrates one example of a possible targeted gene expression sequencing data analysis workflow.

FIG. 5 provides a schematic workflow 500, which is an expansion of the sequencing data analysis workflow 190 of FIG. 1, in accordance with various embodiments. It should be appreciated that the methodologies described in the workflow 500 of FIG. 5 and accompanying disclosure can be implemented independently of the methodologies for generating single cell targeted gene expression sequencing data described in FIG. 1. Therefore, FIG. 5 can be implemented independently of the sequencing data generating workflow as long as it is capable of sufficiently analyzing single cell targeted gene expression sequencing data, in accordance with various embodiments.

Accordingly, in accordance with various embodiments, systems and methods within the disclosure can further include a data analysis workflow (i.e., a computational pipeline) for analyzing the sequencing data generated by the single cell targeted gene expression sequencing workflow described above. The data analysis workflow can include one or more of the following analysis steps. Not all the steps within the disclosure of FIG. 5 need to be utilized as a group. Therefore, some of the steps within FIG. 5 are capable of independently performing the necessary data analysis as part of the various embodiments disclosed herein. Accordingly, it is understood that, certain steps within the disclosure can be used either independently or in combination with other steps within the disclosure, while certain other steps within the disclosure can only be used in combination with certain other steps within the disclosure. Further, one or more of the steps or filters described below, presumably defaulted to be utilized as part of the computational pipeline for analyzing the single cell targeted gene expression sequencing data, can also not be utilized per user input. It is understood that the reverse is also contemplated. It is further understood that additional steps for analyzing the sequencing data generated by the single cell targeted gene expression sequencing workflow are also contemplated as part of the computational pipeline within the disclosure.

Barcode Processing

The workflow 500 can comprise, at step 502, processing the barcodes in the single cell targeted gene expression sequencing data for fixing the occasional sequencing error in the barcodes so that the sequenced fragments can be associated with the original barcodes, thus improving the data quality. Detail related to the barcode processing and correction as part of the various embodiments disclosed herein is provided below.

In accordance with various embodiments, the barcode sequence can be between about 2 bp to about 25 bp. In accordance with various other embodiments, the barcode sequence can be between about 5 bp and 20 bp. In accordance with various preferred embodiments, the barcode sequence can be between about 10 bp and 16 bp. The length of the barcode sequence can affect the number of unique barcodes present in the sequencing library. Accordingly, it is understood that barcode sequences shorter than 10 bp can be selected in accordance with various embodiments herein, provided that the read sequence data from multiple cells are not associated with the same barcode because of severe lack of diversity caused by a shorter length of the barcode sequence. The barcode sequence can be obtained from the “12” index read and is read as part of the I2 reaction. Accordingly, it is understood that barcode sequences longer than 16 bp can be selected in accordance with various embodiments herein, provided that the barcode sequence length is within the limits of the I2 index read and reaction, and that it can be sequenced on a sequencer within the various embodiments herein. The barcode processing step can include checking each barcode sequence against a “whitelist” of correct barcode sequences. The barcode processing step can further include counting the frequency of each whitelist barcode. The barcode processing step can also include various barcode correction steps as part of the various embodiments disclosed herein. For example, one may attempt to correct the barcodes that are not included on the whitelist by finding all the whitelisted barcodes that are within 2 differences (Hamming distance <=2) of the observed sequence, and then scoring them based on the abundance of that barcode in the read data and quality value of the incorrect bases. As another example, an observed barcode that is not present in the whitelist can be corrected to a whitelist barcode if it has >90% probability of being the real barcode.

Alignment

The workflow 500 can comprise, at step 504, aligning the read sequences (also referred to as the “reads”) to a reference sequence. One of more sub-steps can be utilized for trimming off adapter sequences, primer oligo sequences, or both in the read sequence before the read sequence is aligned to the reference genome. Detail related to trimming and aligning the read sequences as part of the various embodiments disclosed herein is provided below.

In the alignment step of the various embodiments herein, a reference-based analysis is performed by aligning the read sequences (also referred to as the “reads”) to a reference sequence. The reference sequence of the various embodiments herein can include a reference genome sequence and its associated genome annotation, which includes gene and transcript coordinates. The genome sequences and annotations (e.g., gene and transcript coordinates) of various embodiments herein can be obtained from reputable, well-established consortia, including but not limited to, NCBI, GENCODE, Ensembl, and ENCODE. In various embodiments, the reference sequence can include single species and/or multi-species reference sequences. In various embodiments, systems and methods within the disclosure can also provide pre-built single and multi-species reference sequences. In various embodiments, the pre-built reference sequences can include information and files related to regulatory regions including, but not limited to, annotation of promoter, enhancer, CTCF binding sites, and DNase hypersensitivity sites. In various embodiments, systems and methods within the disclosure can also provide building custom reference sequences that are not pre-built.

The alignment step may further include various sub-steps. For example, in various embodiments, the alignment step may include a sub-step that trims off the adapter, primer oligo sequence, or both in the read sequence before the read sequence is aligned to the reference genome. In various embodiments, the 3′ end of a read (i.e., the end of the read sequence) is presumed to contain the reverse complement of the primer sequence if the read sequence length is greater than the length of the genomic fragment. In such an event, the alignment step within various embodiments herein may include a sub-step that can identify the reverse complement of the primer sequence at the end of each read and trims off such sequence from the read sequence before the read sequence is aligned to the reference genome. In various embodiments, the cutadapt tool can be used to identify and trim off the reverse complement of the primer sequence from the read sequence prior to alignment.

The trimmed read-pairs described above can then be aligned to a specified genome reference using various methods in accordance with various embodiments herein. For example, in various embodiments, BWA-MEM with default parameters can be used to align all the trimmed read-pairs to a specified reference. In various embodiments, such default parameter can be bwa mem-t<num_threads>-M-R<read_group_header><ref_fasta><R1.fastq><R2.fastq>. BWA-MEM does not align read sequences that are less than 25 bp. Accordingly, when BWA-MEM is used with various embodiments herein, the unaligned reads are included in the BAM output and flagged as unmapped. In various embodiments, the unmapped sequences may not be used in downstream analysis as they may be incapable of contributing to any information due to lack of mapping.

Gene Annotation Processing

The workflow 500 can comprise, at step 506, annotating the individual RNA fragment reads as exonic, intronic, intergenic, and by whether they align to the reference genome with high confidence. In various embodiments, a fragment read is annotated as exonic if at least 50% of it intersects one or more exons contained within the transcript of a gene (“the overlapping gene”). In various embodiments, a fragment read is annotated as intronic if it is non-exonic and intersects the intron of a gene. In various embodiments, a fragment read is annotated as transcriptomic if it is exonic, the orientation of the alignment is to the annotated sense strand of the overlapping gene, and any putative RNA splice junctions in the alignment match the annotated RNA splice junctions of one or more transcripts from the overlapping gene. In various embodiments, a fragment read is annotated as confidently mapped and transcriptomic if it can be annotated as transcriptomic for exactly one overlapping gene.

Unique Molecule Processing

The workflow 500 can comprise, at step 508, counting confidently mapped transcriptomic sequence reads with the same barcode, UMI and gene annotation. In various embodiments, if two groups of sequence reads have the same barcode and gene annotation, but their UMIs differ by a single base, then the UMI of the smaller sequence read group is corrected with the UMI of the larger sequence read group. A UMI corrected in this manner may be referred to as a “corrected UMI.” Ties can be broken by any deterministic method (e.g., by choosing the lexicographically smaller UMI) or ignored by leaving both UMIs uncorrected. In various embodiments, if two or more groups of sequence reads have the same barcode and UMI (such as a corrected UMI or an uncorrected UMI), but different gene annotations, the gene annotation with the most supporting reads is kept for UMI counting and the other read groups are discarded. Ties can be broken by any deterministic method (e.g., by choosing the lexicographically smaller gene name) or ignored by discarding both or neither of the read groups.

Targeted Unique Molecule Filtering

The workflow 500 can comprise, at step 510, filtering out groups of sequence reads with the same barcode, UMI (such as a corrected UMI or an uncorrected UMI), and gene annotation when the groups do not include a dynamically set threshold number of sequence reads. This is done to eliminate sequence reads of PCR artifacts. The details of how this is accomplished will be discussed in detail below.

Cell Calling

The workflow 500 can comprise, at step 512, a cell calling analysis that includes associating a subset of barcodes observed in the library to the cells loaded from the sample. Identification of these cell barcodes can allow one to then analyze the variation in data at a single cell resolution. The process may further include correction of gel bead artifacts, such as gel bead multiples (where a cell shares more than one barcoded gel bead) and barcode multiplets (which occurs when a cell associated gel bead has more than one barcode). In some embodiments, the steps associated with cell calling and correction of gel bead artifacts are utilized together for performing the necessary analysis as part of the various embodiments herein.

Feature-Barcode Matrix Generation

The workflow 500 can comprise, at step 514, generating a feature-barcode matrix that summarizes the gene expression counts per each cell. The feature-barcode contains one row per feature and one column for each cell-associated barcode, as determined by the cell calling step. The set of features may comprise the entire set of genes against which gene annotation is performed, or only the subset of on-target genes. If counts of other analytes are also measured, those analytes may also be included as features, i.e., rows. Each matrix entry indicates the count of unique molecules (i.e., passing the unique molecule filtering steps above) observed with the given cell barcode and assigned the given feature.

Dimensionality Reduction, Clustering, and T-SNE Projection

The workflow 500 can comprise, at step 516, various dimensionality reduction, clustering and t-SNE projection tools. Dimensionality reduction tools of the various embodiments herein are utilized to reduce the number of random variables under consideration by obtaining a smaller set of transformed variables. In accordance with various embodiments herein, clustering tools can be utilized to assign objects of the various embodiments herein to homogeneous groups (called clusters) while ensuring that objects in different groups are not similar. T-SNE projection tools of the various embodiments herein can include an algorithm for visualization of the data of the various embodiments herein. In accordance with various embodiments, systems and methods within the disclosure can further include dimensionality reduction, clustering and t-SNE projection tools. In some embodiments, the analysis associated with dimensionality reduction, clustering, and t-SNE projection for visualization are utilized together for performing the necessary analysis as part of the various embodiments herein. Various analysis tools such as Principal Component Analysis (PCA), clustering, and t-SNE projection for visualization that allow one to group and compare a population of cells with another, detail related to which are provided below.

Biological discovery is often aided by visualization tools that allow one to group and compare a population of cells with another. In order to enable such discovery, various visualization methods within the various embodiments herein, e.g., visualization methods within the Cell Ranger™ Targeted Gene Expression analysis pipeline, can be employed. In various embodiments, such visualization methods can include clustering and T-distributed Stochastic Neighbor Embedding (t-SNE) projection tools.

The systems and methods within the disclosure are directed to identifying differential gene expression. As the data is sparse at single cell resolution, clustering in accordance with various embodiments herein can be used to aggregate data from groups of similar cells.

Various systems and methods of various embodiments herein, e.g., systems and methods of the Cell Ranger™ Targeted Gene Expression analysis pipeline, can be utilized to support dimensionality reduction. Various methods, such as Principal Component Analysis (PCA), can support dimensionality reduction in accordance with various embodiments herein. In various embodiments, before clustering the cells, PCA is run on the normalized filtered feature-barcode matrix to reduce the number of feature (gene) dimensions. This produces a projection of each cell onto the first N components (default N=10). Thus, in various embodiments, the adopted default method of dimensionality reduction is PCA. In accordance with various embodiments herein, each dimensionality reduction method within the disclosure can have an associated data normalization technique that is used prior to the dimensionality reduction step.

In accordance with various embodiments herein, a collection of clustering methods within the disclosure can be employed to accept the dimensionality reduced data. In various embodiments, an optimized implementation of the Barnes Hut TSNE algorithm can be employed to project the dimensionality reduced data into 2-D t-SNE space. In accordance with various embodiments herein, the number of dimensions can be fixed to 15. In various embodiments, the uniform manifold approximation and project (UMAP algorithm) can be employed to project the dimensionality reduced data into UMAP space. In some embodiments within the disclosure, it has been observed that fixing the number of dimensions 10 can sufficiently separate clusters visually and in a biologically meaningful way when tested on peripheral blood mononuclear cells (PBMCs). It is understood that other number of dimensions can be fixed in accordance with various embodiments herein. In various embodiments within the disclosure, the number of dimensions (i.e., dimensions of the reduced matrix) can be fixed at a number less than the number of cell-barcodes and the number of features. In various embodiments, the number of dimensions can be at least 15. In various embodiments, the number of dimensions can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100. It can be understood that higher numbers of dimensions can be computationally costly. Accordingly, computational costs can be determinative of the number of dimensions used. More detail regarding the various methods dimensionality reduction methods described above is provided below.

When PCA is the dimensionality reduction method of the various embodiments herein, the data is normalized to median UMI counts per barcode and log-transformed. In various embodiments, a fast, scalable and memory efficient implementation of IRLBA (Augmented, Implicitly Restarted Lanczos Bidiagonalization Algorithm) can be used that allows in-place centering and feature scaling and produces the transformed matrix along with the principal components (PC) and singular values encoding the variance explained by each PC. Regardless of the dimensionality reduction method of the various embodiments herein, clustering can be used to find clusters in the within the transformed matrix. In various embodiments, k-means clustering is used to produce 2 to 10 clusters for visualization and analysis. In various embodiments, a k-nearest neighbors graph-based clustering method is also provided via community detection using a modularity optimization algorithm. In various embodiments, the modularity optimization algorithm is the Louvain modularity optimization algorithm. The transformed matrix of the various embodiments herein, can be operated on by the t-SNE algorithm with default parameters (e.g., tsne_input_pcs, tsne_perplexity, tsne_theta, tsne_max_dims, tsne_max_iter, tsne_stop_lying_iter, and tsne_mom_switch_iter) and can provide 2-D coordinates for each barcode for visualization with various embodiments herein.

Below is a summary of default parameters of t-SNE algorithm in accordance with various embodiments.

| | | Default | | |
|---|---|---|---|---|
| Parameter | Type | Value | Recommended Range | Description |
| tsne_input_pcs | int | null | Cannot be set higher than the | Subset to top N |
| | | | num_comps parameter, which | principal components |
| | | | is N principal components for | for TSNE. |
| | | | LSA/PCA/PLSA. | |
| tsne_perplexity | int | 30 | 30-50 | TSNE perplexity |
| | | | | parameter. |
| tsne_theta | float | 0.5 | Between 0 and 1. | TSNE theta parameter. |
| tsne_max_dims | int | 2 | 2 or 3. | Maximum number of |
| | | | | TSNE output |
| | | | | dimensions. |
| tsne_max_iter | int | 1000 | 1000-10000 | Number of total TSNE |
| | | | | iterations. |
| tsne_stop_lying_iter | int | 250 | Cannot be set higher than | Iteration at which |
| | | | tsne_max_iter. | TSNE learning rate is |
| | | | | reduced. |
| tsne_mom_switch_iter | int | 250 | Cannot be set higher than | Iteration at which |
| | | | tsne_max_iter. | TSNE momentum is |
| | | | | reduced. |

It is understood that various clustering methods including, but not limited to, K-Means clustering, affinity propagation, mean-shift, spectral clustering, Ward hierarchical clustering, agglomerative clustering, DBSCAN, OPTICS, Gaussian mixture models, Birch clustering, and k-medoids clustering, and visualization approaches can be utilized in accordance with various embodiments herein. It is understood that each clustering method may have various tradeoffs. Accordingly, in various embodiments, selection of a clustering method can be made based on whether the clusters make biological sense with known, well-studied sample types (e.g., PBMCs), i.e., whether the clusters generated using a particular clustering method make sense with validation on known biology. Below is a non-limiting summary of dimensionality reduction techniques and associated clustering and visualization approaches in accordance with various embodiments herein.

| Dimensionality Reduction | Clustering | Visualization |
|---|---|---|
| PCA | K-means, graph-clustering | TSNE, UMAP |

Differential Expression Analysis

The workflow 500 can comprise, at step 518, a differential expression analysis that performs differential analysis to identify genes whose expression is specific to each cluster, Cell Ranger tests, for each gene and each cluster, whether the in-cluster mean differs from the out-of-cluster mean.

In order to find differentially expressed genes between groups of cells, systems and methods disclosed herein can use the quick and simple method sSeq (Yu, D., Huber, W. & Vitek, O. “Shrinkage estimation of dispersion in Negative Binomial models for RNA-seq experiments with small sample size” Bioinformatics 29, 1275-1282 (2013)), which employs a negative binomial exact test. When the counts become large, systems and methods disclosed herein switches to the fast asymptotic beta test used in edgeR (Robinson, M. D. & Smyth, G. K. “Small-sample estimation of negative binomial dispersion, with applications to SAGE data” Biostatistics 9, 321-332 (2007)). For each cluster, the algorithm is run on that cluster versus all other cells, yielding a list of genes that are differentially expressed in that cluster relative to the rest of the sample. Each of these references is incorporated herein by reference in its entirety for all purposes.

The implementation of the systems and methods disclosed herein differs slightly from that in the sSeq paper where the authors recommend using DESeq's geometric mean-based definition of library size. The systems and methods disclosed herein, instead, computes relative library size as the total UMI counts for each cell divided by the median UMI counts per cell. As with sSeq, normalization is implicit in that the per-cell library-size parameter is incorporated as a factor in the exact-test probability calculations.

### Conventional Chimeric Molecule Filtering Techniques

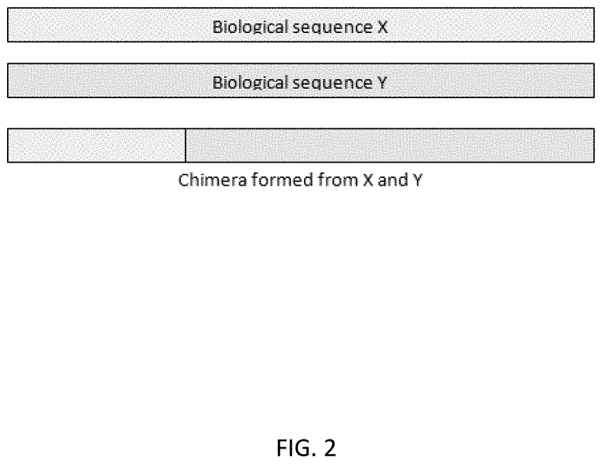

FIG. 2 is an illustration of a chimeric molecule (or chimera), in accordance with various embodiments. As shown in FIG. 2, a chimeric molecule may take portions of a first biological sequence (denoted “X”) and a second biological sequence (denoted “Y”). The chimeric molecule then consists of a hybrid formed from at least a portion of sequence X and at least a portion of sequence Y. Sequence X and sequence Y may each comprise a nucleic acid sequence, such as a DNA sequence or RNA sequence.

Generally, PCR created chimeric molecules are less amplified than molecules from the original library as chimeric molecules only undergo a portion of the PCR amplification cycles. FIG. 3 illustrates PCR amplification effects on pooled libraries with chimeric molecules, in accordance with various embodiments. As shown in FIG. 3, an amplified pooled library of genomic fragments with distinct UMIs 302 is created and then sequenced resulting in a plurality of genomic sequence reads that contain regular genomic fragments with distinct UMI sequences 304 and chimeric molecule sequences 306.

A number of conventional filtering methods have used this observation combined with barcoding information to remove chimeric molecule sequences from a sequencing dataset. For example, in one conventional method, when two molecules are observed with identical cell-barcode and UMI (but different genes), the molecule with fewer supporting sequence reads is automatically discarded as a likely from a chimeric molecule. However, the enrichment that is performed as part of a targeted gene expression analysis workflow can result in a chimeric molecule (cell-barcode A, UMI A, on-target gene B) becoming more abundant than a true molecule with (cell-barcode A, UMI A, off-target gene A). For this reason, that method is not sufficient for use in a targeted gene expression analysis workflow.

Another conventional method considers only the counts of sequencing reads supporting each putative molecule (i.e., combination of barcode, UMI, and gene). (This method may be used after using the method directly above to select a single gene for each barcode-UMI combination). When a plot of the number of supporting reads of each putative molecule is observed as a bimodal distribution, the lower mode of the distribution likely represents artifact molecules, and the upper mode likely represents true molecules. The algorithm fits two negative binomial distributions to statistically distinguish between the two modes. Fragment sequence reads comprising putative molecules in the upper mode are retained, while the fragment sequence reads comprising putative molecules in the lower mode are discarded. In practice, the high number of degrees of freedom in the fitting procedure (5 parameters; 2 for each negative binomial distribution and 1 mixing proportion) make the results of this method difficult to predict.

Another conventional method to remove very low-abundance molecules that are suspected to be chimeric is data subsampling. By randomly choosing a subset of the sequencing reads and discarding the rest, molecules represented/supported by very few reads will be preferentially discarded, i.e., all of the representative reads will have been discarded. However, the randomness introduced by subsampling methods is undesirable, and it is not obvious how much of the data should be discarded in order to remove most of the artifacts without removing too many true molecules.

### New Chimeric Molecule Filtering for Targeted Gene Expression Analysis

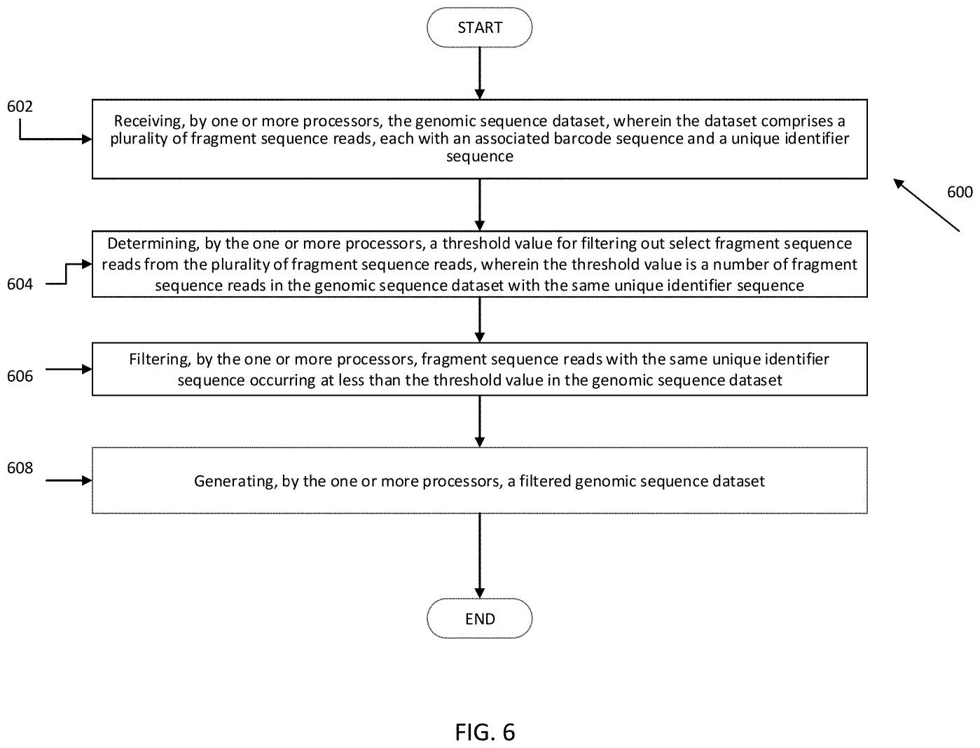

Referring now to FIG. 6, a flowchart illustrating a non-limiting example method 600 for filtering out erroneous sequence reads from a genomic sequence dataset is disclosed, in accordance with various embodiments, in accordance with various embodiments. The method 600 can comprise, at step 602, receiving the genomic sequence dataset by one or more processors. The dataset includes a plurality of fragment sequence reads each associated with a barcode sequence and a unique identifier sequence (i.e., UMI).

The method can comprise, at step 604, determining a threshold value for filtering out select fragment sequence reads, using the one or more processors. The threshold value is a number of fragment sequence reads in the genomic sequence dataset with the same unique identifier sequence. That is, the threshold value is a number that represents a cut off number of fragment sequence reads observed for any given UMI within a genomic sequence dataset.

In various embodiments, the threshold value is a dynamic threshold that is calculated as a product of a dynamic quantile modifier (that is derived from the genomic sequence dataset) and a pre-set multiplier, rounded up to the nearest integer value.

In various embodiments, the dynamic quantile modifier is a number of fragment sequence reads registered at a greater than 90th percentile rank in a frequency distribution plot of fragment sequence reads with the same unique identifier sequence (See FIG. 4) found in a genomic sequence dataset. That is, the dynamic quantile modifier would be a count of fragment sequence reads that represents an upper bound for 10% of UMIs of a frequency distribution plot of the number of sequence reads per UMI found in a genomic sequence dataset.

In various embodiments, the dynamic quantile modifier is a number of fragment sequence reads registered at a greater than about a 50th percentile rank, 55th percentile rank, 60th percentile rank, 65th percentile rank, 70th percentile rank, 75th percentile rank, 80th percentile rank, 85th percentile rank, 95th percentile rank, or 99th percentile rank in a frequency distribution plot of fragment sequence reads with the same unique identifier sequence found in a genomic sequence dataset.

In various embodiments, the pre-set multiplier is a value between about 0.005 and about 0.05, about 0.025 and about 0.1, or about 0.001 and about 0.5. In various embodiments, the pre-set multiplier is inversely correlated with the dynamic quantile modifier described above.

The method can comprise, at step 606, filtering fragment sequence reads with the same unique identifier sequence occurring at less than the threshold value in the genomic sequence dataset, using the one or more processors. For example, if the threshold value is determined to be 5, any UMIs found in fewer than 5 fragment sequence reads are considered to have been sequenced from an erroneous fragment (e.g., chimeric molecule, PCR artifact, etc.) and are therefore removed from the genomic sequence dataset. This is illustrated in FIG. 4 where a threshold value 202 was set at 4 reads and all the UMIs found in fewer than 4 fragment sequence reads (to the left of the threshold value 202 line) were filtered out as being from an erroneous fragment (e.g., chimeric molecule, PCR artifact, etc.).

The method can comprise, at step 608, generating a filtered genomic sequence dataset, using the one or more processors.

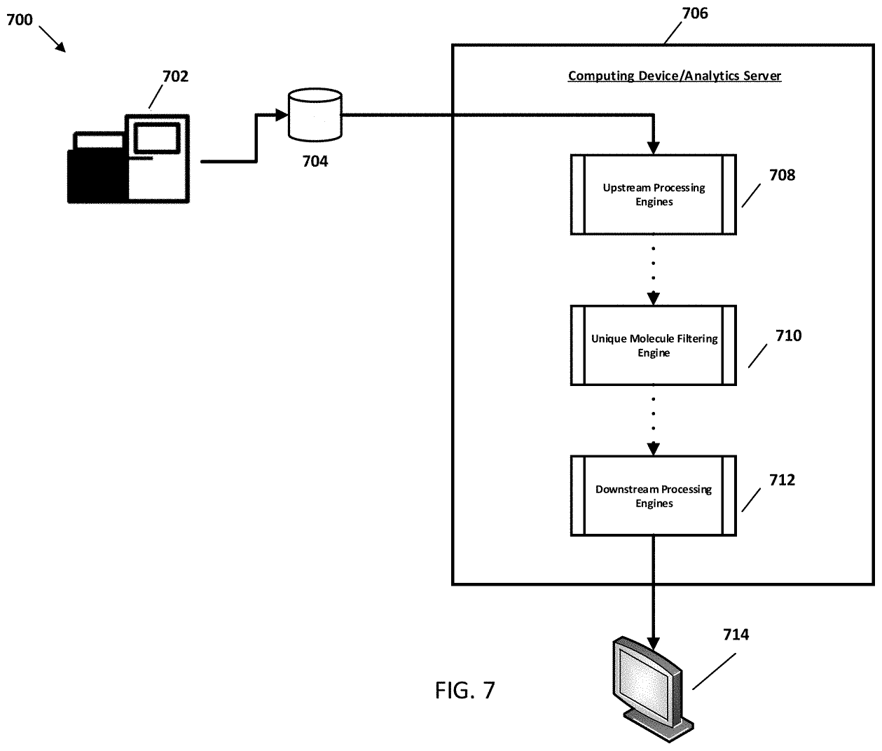

In accordance with various embodiments, FIG. 7 illustrates a non-limiting example system 700 for filtering out erroneous sequence reads from a genomic sequence dataset, in accordance with various embodiments. The system 700 includes a genomic sequence analyzer 702, a data storage unit 704, a computing device/analytics server 706, and a display 714.

The genomic sequence analyzer 702 can be communicatively connected to the data storage unit 704 by way of a serial bus (if both form an integrated instrument platform) or by way of a network connection (if both are distributed/separate devices). The genomic sequence analyzer 702 can be configured to process, analyze and generate one or more genomic sequence datasets from a sample, such as the targeted gene expression fragment libraries of the various embodiments herein. Each fragment in the library includes an associated barcode and unique identifier sequence (i.e., UMI). In various embodiments, the genomic sequence analyzer 702 can be a next-generation sequencing platform or sequencer such as the Illumina® sequencer, MiSeq™, NextSeq™ 500/550 (High Output), HiSeq 2500™ (Rapid Run), HiSeq™ 3000/4000, and NovaSeq.

In various embodiments, the generated genomic sequence datasets can then be stored in the data storage unit 704 for subsequent processing. In various embodiments, one or more raw genomic sequence datasets can also be stored in the data storage unit 704 prior to processing and analyzing. Accordingly, in various embodiments, the data storage unit 704 can be configured to store one or more genomic sequence datasets, e.g., the genomic sequence datasets of the various embodiments herein that includes a plurality of fragment sequence reads with their associated barcodes and unique identifier sequences. In various embodiments, the processed and analyzed genomic sequence datasets can be fed to the computing device/analytics server 706 in real-time for further downstream analysis.

In various embodiments, the data storage unit 704 is communicatively connected to the computing device/analytics server 706. In various embodiments, the data storage unit 704 and the computing device/analytics server 706 can be part of an integrated apparatus. In various embodiments, the data storage unit 704 can be hosted by a different device than the computing device/analytics server 706. In various embodiments, the data storage unit 704 and the computing device/analytics server 706 can be part of a distributed network system. In various embodiments, the computing device/analytics server 706 can be communicatively connected to the data storage unit 704 via a network connection that can be either a “hardwired” physical network connection (e.g., Internet, LAN, WAN, VPN, etc.) or a wireless network connection (e.g., Wi-Fi, WLAN, etc.). In various embodiments, the computing device/analytics server 706 can be a workstation, mainframe computer, distributed computing node (part of a “cloud computing” or distributed networking system), personal computer, mobile device, etc.

In various embodiments, the computing device/analytics sever 706 is configured to host one or more upstream data processing engines 708, a Unique Molecule Filtering Engine 710, and one or more downstream data processing engines 712.

Examples of upstream data processing engines 708 can include, but are not limited to: alignment engine, cell barcode processing engine (for correcting sequencing barcode sequencing errors), alignment engine (for aligning the fragment sequence reads to a reference genome), annotation engine (for annotating each of the aligned fragment sequence reds with relevant information), etc.

The Unique Molecule Filtering Engine 710 can be configured to receive one or more genomic sequence datasets that are stored in the data storage unit 704. The genomic sequence datasets are comprised for a plurality of fragment sequence reads (generated from the sequencing of a fragment library, for example, a targeted gene expression fragment library), each with an associated barcode sequence and a unique identifier sequence (i.e., UMI). In various embodiments, the Unique Molecule Filtering Engine 710 can be configured to receive processed and analyzed genomic sequence datasets from the genomic sequence analyzer 702 in real-time.

In various embodiments, the Unique Molecule Filtering Engine 710 can be configured to determine a threshold value for filtering out select fragment sequence reads from the genomic sequence dataset. In various embodiments, the threshold value is a number of fragment sequence reads in the genomic sequenced dataset with the same unique identifier sequence.

In various embodiments, the threshold value is a dynamic threshold that is calculated as a product of a dynamic quantile modifier (that is derived from the genomic sequence dataset) and a pre-set multiplier.

In various embodiments, the dynamic quantile modifier is a number of fragment sequence reads registered at a greater than 90th percentile rank in a frequency distribution plot of fragment sequence reads with the same unique identifier sequence (See FIG. 4) found in a genomic sequence dataset. That is, the dynamic quantile modifier would be the largest observed count C of fragment sequence reads such that fewer than 10% of counts in the frequency distribution lie above C.

In various embodiments, the dynamic quantile modifier is a number of fragment sequence reads registered at a greater than about a 50th percentile rank, 55th percentile rank, 60th percentile rank, 65th percentile rank, 70th percentile rank, 75th percentile rank, 80th percentile rank, 85th percentile rank, 95th percentile rank, or a 99th percentile rank in a frequency distribution plot of fragment sequence reads with the same unique identifier sequence found in a genomic sequence dataset.

In various embodiments, the pre-set multiplier is a value between about 0.005 and about 0.05, about 0.025 and about 0.1, or about 0.001 and about 0.5. In various embodiments, the pre-set multiplier is inversely correlated with the dynamic quantile modifier described above.

In various embodiments, the Unique Molecule Filtering Engine 710 can further be configured to filter fragment sequence reads with the same unique identifier sequence occurring at less than the threshold value in the genomic sequence dataset. In various embodiments, the Unique Molecule Filtering Engine 710 can further be configured to filter fragment sequence reads with the same unique identifier sequence occurring at less than the threshold value in the genomic sequence dataset. For example, as discussed above, if the threshold value is determined to be 5, any UMIs found in 5 or less fragment sequence reads are considered to have been sequenced from an erroneous fragment (e.g., chimeric molecule, PCR artifact, etc.) and are therefore removed from the genomic sequence dataset.

Lastly, in various embodiments, the Unique Molecule Filtering Engine 710 can further be configured to generate a filtered genomic sequence dataset.

The filtered genomic sequence dataset can then be further processed by one or more downstream data processing engines 712. Examples of downstream data processing engines 712 can include, but are not limited to: cell calling engine (for grouping fragment sequence reads as being from a unique cell), feature barcode matrix engine (for creating a feature barcode matrix), differential analysis engine (for identifying genes whose expression is specific to each cell cluster), etc.

After the downstream processing has been performed and an output of the results can be displayed as a result or summary on a display or client terminal 714 that is communicatively connected to the computing device/analytics server 706. In various embodiments, the display or client terminal 714 can be a thin client computing device. In various embodiments, the display or client terminal 714 can be a personal computing device having a web browser (e.g., INTERNET EXPLORER™, FIREFOX™, SAFARI™, etc.) that can be used to control the operation of the genomic sequence analyzer 702, data store 704, upstream data processing engines 708, Unique Molecule Filtering Engine 710, and the downstream data processing engines 712.

### Experimental Results

Chimeric molecule filtering for a targeted gene expression library can be assessed quantitatively by comparing the targeted sequencing library to an untargeted “parent” library, i.e., sequencing data collected from the original pre-enrichment gene expression library.

As discussed above, the unique molecule filtering step can be expected to remove chimeric molecules from the non-targeted library, so the remaining set of molecules can be taken as “true” for the purpose of comparison. These molecules can be compared to the initial set of unique molecules obtained from the targeted library, many of which are expected to have an identical barcode, UMI, gene combination as a unique molecule from the parent library, since the targeted library is merely an enriched version of the parent library. These unique molecules in the targeted library with matching molecules from the parent library are considered “confirmed.” Unique molecules from the targeted library whose barcode, UMI combination is identical to a unique molecule from the parent library, but whose gene annotation is different, can be labeled “chimeric.” Finally, unique molecules in the targeted library whose barcode, UMI combination does not appear in the set of unique molecules from the parent library are labeled “unconfirmed,” as their chimera status cannot be reliably determined.

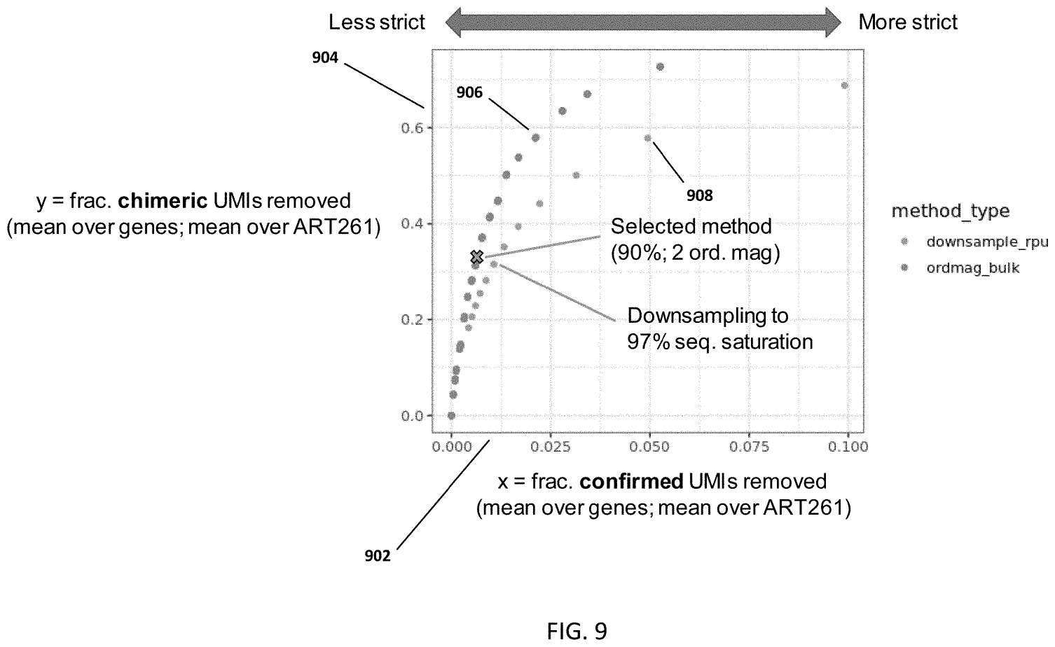

For a given molecule filtering procedure, one can measure two quantities: the fraction of confirmed molecules removed by the procedure (the “false positive rate”), the fraction of chimeric molecules removed by the procedure (the “true positive rate”). A good procedure is one that, for its given false positive rate, has a high true positive rate among procedures considered. FIG. 9 shows the performance of two molecule filtering strategies: the methods described herein (906), and data subsampling (908), in accordance with various embodiments. The x-axis 902 shows false positive rate, and the y-axis 904 shows true positive rate. As shown in the figure, if each method is adjusted to the same average false positive rate (x-axis 902), the proposed method can be expected to achieve approximately twice the average true positive rate (y-axis 904).

Each plotted point represents the average performance of a single method specification across many samples. The proposed method has two parameters that determine its specification: a percentile (90) and multiplier (0.01) that determine how the dynamic threshold is computed. The data subsampling method in this case is specified by the desired mean of the frequency distribution of sequence reads per UMI after subsampling (which may remove some or all reads from a given UMI).

### Computer System

In various embodiments, the methods for filtering out erroneous sequence reads from a genomic sequence dataset is can be implemented via computer software or hardware. That is, as depicted in FIG. 7, the methods disclosed herein can be implemented on a computing device/analytics sever 706 that includes upstream data processing engines 708, a Unique Molecule Filtering Engine 710, and downstream data processing engines 712. In various embodiments, the computing device/analytics sever 706 can be communicatively connected to a genomic sequence analyzer 702, a data store 704, and a display or client terminal 714, via a direct connection or through an internet connection.

It should be appreciated that the various engines depicted in FIG. 7 can be combined or collapsed into a single engine, component or module, depending on the requirements of the particular application or system architecture. Moreover, in various embodiments, the upstream data processing engines 708, Unique Molecule Filtering Engine 710, and the downstream data processing engines 712, can comprise additional engines or components as needed by the particular application or system architecture.

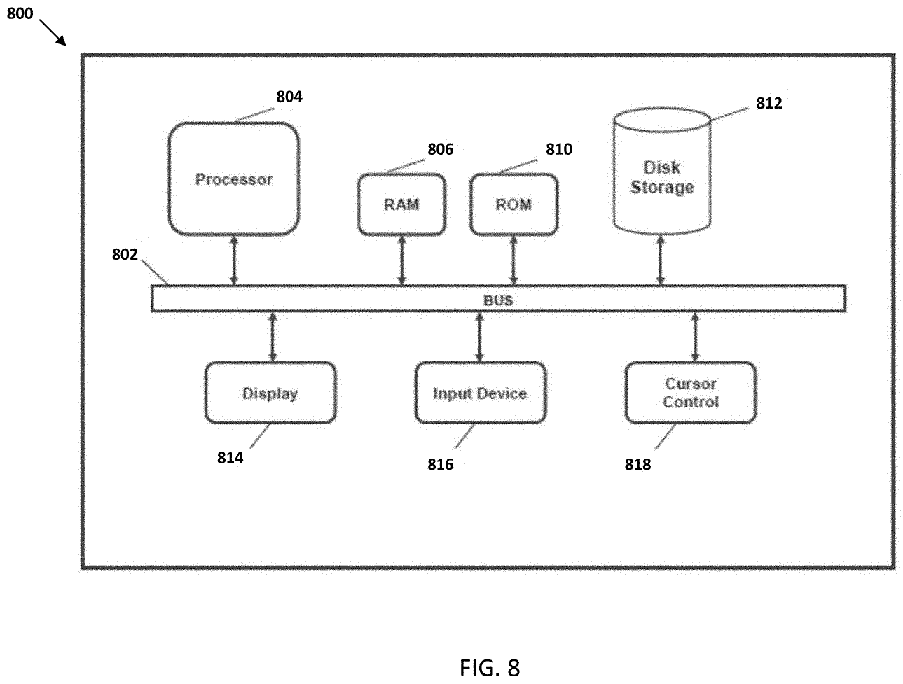

FIG. 8 is a block diagram that illustrates a computer system 800, upon which embodiments of the present teachings may be implemented. In various embodiments of the present teachings, computer system 800 can include a bus 802 or other communication mechanism for communicating information, and a processor 804 coupled with bus 802 for processing information. In various embodiments, computer system 800 can also include a memory, which can be a random access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 810 or other static storage device coupled to bus 802 for storing static information and instructions for processor 804. A storage device 812, such as a magnetic disk or optical disk, can be provided and coupled to bus 802 for storing information and instructions.

In various embodiments, computer system 800 can be coupled via bus 802 to a display 814, such as a cathode ray tube (CRT) or liquid crystal display (LCD), for displaying information to a computer user. An input device 816, including alphanumeric and other keys, can be coupled to bus 802 for communicating information and command selections to processor 804. Another type of user input device is a cursor control 818, such as a mouse, a trackball or cursor direction keys for communicating direction information and command selections to processor 804 and for controlling cursor movement on display 814. This input device 816 typically has two degrees of freedom in two axes, a first axis (i.e., x) and a second axis (i.e., y), that allows the device to specify positions in a plane. However, it should be understood that input devices 816 allowing for 3 dimensional (x, y and z) cursor movement are also contemplated herein.

Consistent with certain implementations of the present teachings, results can be provided by computer system 800 in response to processor 804 executing one or more sequences of one or more instructions contained in memory 806. Such instructions can be read into memory 806 from another computer-readable medium or computer-readable storage medium, such as storage device 812. Execution of the sequences of instructions contained in memory 806 can cause processor 804 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 812. Examples of volatile media can include, but are not limited to, dynamic memory, such as memory 806. Examples of transmission media can include, but are not limited to, coaxial cables, copper wire, and fiber optics, including the wires that comprise bus 802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flowcharts, diagrams and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, memory components 806/810/812 and user input provided via input device 816.

### RECITATION OF EMBODIMENTS

Embodiment 1. A method for filtering out erroneous sequence reads from a genomic sequence dataset, comprising:

receiving, by one or more processors, the genomic sequence dataset, wherein the dataset comprises a plurality of fragment sequence reads, each with an associated barcode sequence and a unique identifier sequence;

determining, by the one or more processors, a threshold value for filtering out select fragment sequence reads from the genomic sequence dataset, wherein the threshold value is a number of fragment sequence reads in the genomic sequence dataset with the same unique identifier sequence;

filtering, by the one or more processors, fragment sequence reads with the same unique identifier sequence occurring at less than the threshold value in the genomic sequence dataset; and

generating, by the one or more processors, a filtered genomic sequence dataset.

Embodiment 2. The method of Embodiment 1, wherein the threshold value is a product of a dynamic quantile modifier derived from the genomic sequence dataset and a pre-set multiplier.

Embodiment 3. The method of Embodiment 2, wherein the dynamic quantile modifier is a number of fragment sequence reads registered at a greater than a 90th percentile rank of a frequency distribution plot of fragment sequence reads with a same unique identifier sequence found in the genomic sequence dataset.

Embodiment 4. The method of Embodiment 2, wherein the dynamic quantile modifier is a number of fragment sequence reads registered at a greater than a 95th percentile rank of a frequency distribution plot of fragment sequence reads with a same unique identifier sequence found in the genomic sequence dataset.

Embodiment 5. The method of Embodiment 2, wherein the dynamic quantile modifier is a number of fragment sequence reads registered at a greater than a 75th percentile rank of a frequency distribution plot of fragment sequence reads with a same unique identifier sequence found in the genomic sequence dataset.

Embodiment 6. The method of Embodiment 2, wherein the dynamic quantile modifier is a number of fragment sequence reads registered at a greater than a 50th percentile rank of a frequency distribution plot of fragment sequence reads with a same unique identifier sequence found in the genomic sequence dataset.

Embodiment 7. The method of any one of Embodiments 2-6, wherein the pre-set multiplier is between about 0.005 and about 0.05.

Embodiment 8. The method of any one of Embodiments 2-6, wherein the pre-set multiplier is between about 0.025 and about 0.1.

Embodiment 9. The method of any one of Embodiments 2-6, wherein the pre-set multiplier is between about 0.001 and about 0.5.

Embodiment 10. A non-transitory computer-readable medium in which a program is stored for causing a computer to perform a method for filtering out erroneous sequence reads from a genomic sequence dataset, comprising:

receiving, by one or more processors, the genomic sequence dataset, wherein the dataset comprises a plurality of fragment sequence reads, each with an associated barcode sequence and a unique identifier sequence;

determining, by the one or more processors, a threshold value for filtering out select fragment sequence reads from the genomic sequence dataset, wherein the threshold value is a number of fragment sequence reads in the genomic sequence dataset with the same unique identifier sequence;

filtering, by the one or more processors, fragment sequence reads with the same unique identifier sequence occurring at less than the threshold value in the genomic sequence dataset; and

generating, by the one or more processors, a filtered genomic sequence dataset.

Embodiment 11. A system for filtering out erroneous sequence reads from a genomic sequence dataset, comprising:

a data store configured to store the genomic sequence dataset comprising a plurality of fragment sequence reads, each with an associated barcode sequence and a unique identifier sequence; and

a computing device communicatively connected to the data store, comprising,

a unique molecule filtering engine configured to:

receive the genomic sequence dataset,

determine a threshold value for filtering out select fragment sequence reads from the genomic sequence dataset, wherein the threshold value is a number of fragment sequence reads in the genomic sequence dataset with the same unique identifier sequence, and

filter fragment sequence reads with the same unique identifier sequence occurring at less than the threshold value in the genomic sequence dataset, and

generate a filtered genomic sequence dataset.

Embodiment 12. The system of Embodiment 11, wherein the threshold value is a product of a dynamic quantile modifier derived from the genomic sequence dataset and a pre-set multiplier.

Embodiment 13. The system of Embodiment 12, wherein the dynamic quantile modifier is a number of fragment sequence reads registered at a greater than a 90th percentile rank of a frequency distribution plot of fragment sequence reads with a same unique identifier sequence found in the genomic sequence dataset.

Embodiment 14. The system of Embodiment 12, wherein the dynamic quantile modifier is a number of fragment sequence reads registered at a greater than a 95th percentile rank of a frequency distribution plot of fragment sequence reads with a same unique identifier sequence found in the genomic sequence dataset.

Embodiment 15. The system of Embodiment 12, wherein the dynamic quantile modifier is a number of fragment sequence reads registered at a greater than a 75th percentile rank of a frequency distribution plot of fragment sequence reads with a same unique identifier sequence found in the genomic sequence dataset.

Embodiment 16. The system of Embodiment 12, wherein the dynamic quantile modifier is a number of fragment sequence reads registered at a greater than a 50th percentile rank of a frequency distribution plot of fragment sequence reads with a same unique identifier sequence found in the genomic sequence dataset.

Embodiment 17. The system of any one of Embodiments 12-16, wherein the pre-set multiplier is between about 0.005 and about 0.05.

Embodiment 18. The system of any one of Embodiments 12-16, wherein the pre-set multiplier is between about 0.025 and about 0.1.

Embodiment 19. The system of any one of Embodiments 12-16, wherein the pre-set multiplier is between about 0.001 and about 0.5.

Embodiment 20. The system of any one of Embodiments 11-19, further including:

one or more upstream processing engines configured to process the genomic sequence data set prior to being received by the unique molecule filtering engine.

Embodiment 21. The system of any one of Embodiments 11-20, further including:

one or more downstream processing engines configured to process the filtered genomic sequence data set generated by the unique molecule filtering engine.

Embodiment 22. The system of any one of Embodiments 11-21, wherein the data store and the computing device are part of an integrated apparatus.

Embodiment 23. The system of any one of Embodiments 11-22, wherein the data store is hosted by a different device than the computing device.

Embodiment 24. The system of any one of Embodiments 11-23, wherein the data store and the computing device are part of a distributed network system.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

1. A method for filtering out erroneous sequence reads from a genomic sequence dataset, comprising:
receiving, by one or more processors, the genomic sequence dataset, wherein the dataset comprises a plurality of fragment sequence reads, each with an associated barcode sequence and a unique identifier sequence;
determining, by the one or more processors, a threshold value for filtering out select fragment sequence reads from the genomic sequence dataset, wherein the threshold value is a number of fragment sequence reads in the genomic sequence dataset with the same unique identifier sequence;
filtering, by the one or more processors, fragment sequence reads with the same unique identifier sequence occurring at less than the threshold value in the genomic sequence dataset; and
generating, by the one or more processors, a filtered genomic sequence dataset.

2. The method of claim 1, wherein the threshold value is a product of a dynamic quantile modifier derived from the genomic sequence dataset and a pre-set multiplier.

3. The method of claim 2, wherein the dynamic quantile modifier is a number of fragment sequence reads registered at a greater than a 90th percentile rank of a frequency distribution plot of fragment sequence reads with a same unique identifier sequence found in the genomic sequence dataset.

4. The method of claim 2, wherein the dynamic quantile modifier is a number of fragment sequence reads registered at a greater than a 95th percentile rank of a frequency distribution plot of fragment sequence reads with a same unique identifier sequence found in the genomic sequence dataset.

5. The method of claim 2, wherein the dynamic quantile modifier is a number of fragment sequence reads registered at a greater than a 75th percentile rank of a frequency distribution plot of fragment sequence reads with a same unique identifier sequence found in the genomic sequence dataset.

6. The method of claim 2, wherein the dynamic quantile modifier is a number of fragment sequence reads registered at a greater than a 50th percentile rank of a frequency distribution plot of fragment sequence reads with a same unique identifier sequence found in the genomic sequence dataset.

7. The method of claim 2, wherein the pre-set multiplier is between about 0.005 and about 0.05.

8. The method of claim 2, wherein the pre-set multiplier is between about 0.025 and about 0.1.

9. The method of claim 2, wherein the pre-set multiplier is between about 0.001 and about 0.5.

10. A non-transitory computer-readable medium in which a program is stored for causing a computer to perform a method for filtering out erroneous sequence reads from a genomic sequence dataset, comprising:
receiving, by one or more processors, the genomic sequence dataset, wherein the dataset comprises a plurality of fragment sequence reads, each with an associated barcode sequence and a unique identifier sequence;
determining, by the one or more processors, a threshold value for filtering out select fragment sequence reads from the genomic sequence dataset, wherein the threshold value is a number of fragment sequence reads in the genomic sequence dataset with the same unique identifier sequence;
filtering, by the one or more processors, fragment sequence reads with the same unique identifier sequence occurring at less than the threshold value in the genomic sequence dataset; and
generating, by the one or more processors, a filtered genomic sequence dataset.

11. A system for filtering out erroneous sequence reads from a genomic sequence dataset, comprising:
a data store configured to store the genomic sequence dataset comprising a plurality of fragment sequence reads, each with an associated barcode sequence and a unique identifier sequence; and
a computing device communicatively connected to the data store, comprising,
a unique molecule filtering engine configured to:
receive the genomic sequence dataset,
determine a threshold value for filtering out select fragment sequence reads from the genomic sequence dataset, wherein the threshold value is a number of fragment sequence reads in the genomic sequence dataset with the same unique identifier sequence, and
filter fragment sequence reads with the same unique identifier sequence occurring at less than the threshold value in the genomic sequence dataset, and
generate a filtered genomic sequence dataset.

12. The system of claim 11, wherein the threshold value is a product of a dynamic quantile modifier derived from the genomic sequence dataset and a pre-set multiplier.

13. The system of claim 12, wherein the dynamic quantile modifier is a number of fragment sequence reads registered at a greater than a 90th percentile rank of a frequency distribution plot of fragment sequence reads with a same unique identifier sequence found in the genomic sequence dataset.

14. The system of claim 12, wherein the dynamic quantile modifier is a number of fragment sequence reads registered at a greater than a 95th percentile rank of a frequency distribution plot of fragment sequence reads with a same unique identifier sequence found in the genomic sequence dataset.

15. The system of claim 12, wherein the dynamic quantile modifier is a number of fragment sequence reads registered at a greater than a 75th percentile rank of a frequency distribution plot of fragment sequence reads with a same unique identifier sequence found in the genomic sequence dataset.

16. The system of claim 12, wherein the dynamic quantile modifier is a number of fragment sequence reads registered at a greater than a 50th percentile rank of a frequency distribution plot of fragment sequence reads with a same unique identifier sequence found in the genomic sequence dataset.

17. The system of claim 12, wherein the pre-set multiplier is between about 0.005 and about 0.05.

18. The system of claim 12, wherein the pre-set multiplier is between about 0.025 and about 0.1.

19. The system of claim 12, wherein the pre-set multiplier is between about 0.001 and about 0.5.

20. The system of claim 11, further including:
one or more upstream processing engines configured to process the genomic sequence data set prior to being received by the unique molecule filtering engine.

21. The system of claim 11, further including:
one or more downstream processing engines configured to process the filtered genomic sequence data set generated by the unique molecule filtering engine.

22. The system of claim 11, wherein the data store and the computing device are part of an integrated apparatus.

23. The system of claim 11, wherein the data store is hosted by a different device than the computing device.

24. The system of claim 11, wherein the data store and the computing device are part of a distributed network system.
